(19) United States
(12) Patent Application Publication
(10) Pub. No.: US 2021/0298725 A1
(43) Pub. Date: Sep. 30, 2021

(54) ROTATABLE TISSUE SAMPLING DEVICE

(71) Applicant: Gyrus ACMI, Inc. d/b/a Olympus Surgical Technologies America, Westborough, MA (US)

(72) Inventors: Xavier Gonzalez, Woodinville, WA (US); Madeline C. Graham, Woodinville, WA (US)

(21) Appl. No.: 17/215,906

(22) Filed: Mar. 29, 2021

Related U.S. Application Data
(60) Provisional application No. 63/002,886, filed on Mar. 31, 2020.

(51) Int. Cl. A61B 10/02

(52) U.S. Cl. CPC A61B 10/0266; A61B 2010/045; A61B 10/0283

(57) ABSTRACT

Disclosed embodiments include apparatuses, systems, and methods for extracting a tissue sample. In an illustrative embodiment, an apparatus includes a rotatable sampling element having a cylindrical body that defines a receiving chamber configured to receive therein a tissue sample cut from a tissue mass. A cutting apparatus is disposed at a distal end of the cylindrical body to cut the tissue sample from the tissue mass abutting the distal end responsive to rotation of the cylindrical body as it is pressed against the tissue mass. The apparatus also includes a flexible drive shaft having a distal end fixably engaged with a proximal end of the rotatable sampling element. The flexible drive shaft is linearly movable to motivate the rotatable sampling element to press it against the tissue mass and rotatable to impart rotational force to the rotatable sampling element to cause the rotatable sampling element to rotate around the axis.

### PRIORITY CLAIM

The present application claims the priority and benefit of U.S. Provisional Patent Application Ser. No. 63/002,886 filed Mar. 31, 2020, and entitled “ROTATABLE TISSUE SAMPLING DEVICE.”

### FIELD

The present disclosure relates to extraction of a tissue sample from a remote location within a body.

### BACKGROUND

The statements in this section merely provide background information related to the present disclosure and may not constitute prior art.

It is frequently desirable to extract a tissue sample of a lesion or other tissue mass to test the tissue for malignancies or other possible abnormalities. When the lesion is found at a location that is inside of a body, such as may be detected using X-ray, computed tomography, or ultrasound technologies, it may be desirable to extract a sample to be biopsied without invasive procedures.

Needles or similar probes may be inserted into the body and directed toward the lesion to secure a sample without undertaking an invasive procedure. However, it may be difficult to detach a sample of tissue from a mass and to secure the sample for extraction.

### SUMMARY

Disclosed embodiments include apparatuses, systems, and methods for extracting a tissue sample from within a body.

In an illustrative embodiment, an apparatus includes a rotatable sampling element that includes a cylindrical body defining a receiving chamber, where the receiving chamber is configured to receive therein a tissue sample cut from a tissue mass. A cutting apparatus is disposed at a distal end of the cylindrical body to cut the tissue sample from the tissue mass abutting the distal end responsive to rotation of the cylindrical body as the cutting apparatus is pressed against the tissue mass. The apparatus also includes a flexible drive shaft having a distal end fixably engaged with a proximal end of the rotatable sampling element. The flexible drive shaft is linearly movable to motivate the rotatable sampling element along an axis to press the cutting apparatus against the tissue mass and rotatable to impart rotational force to the rotatable sampling element to cause the rotatable sampling element to rotate around the axis.

In another illustrative embodiment, an apparatus includes a rotatable sampling element that includes a cylindrical body defining a receiving chamber, where the receiving chamber is configured to receive therein a tissue sample cut from a tissue mass. A cutting apparatus is disposed at a distal end of the cylindrical body to cut the tissue sample from the tissue mass abutting the distal end responsive to rotation of the cylindrical body as the cutting apparatus is pressed against the tissue mass. The apparatus also includes a flexible drive shaft having a distal end fixably engaged with a proximal end of the rotatable sampling element. The flexible drive shaft is linearly movable to motivate the rotatable sampling element along an axis to press the cutting apparatus against the tissue mass and rotatable to impart rotational force to the rotatable sampling element to cause the rotatable sampling element to rotate around the axis. An actuator handle including a rotatable actuator is mechanically couplable with a proximal end of the flexible drive shaft to impart the rotational force to the flexible drive shaft.

In a further illustrative embodiment, a method includes positioning a rotatable sampling element adjacent to a tissue mass. The rotatable sampling element is advanced and rotated so that a cutting apparatus cuts a tissue sample from the tissue mass. The tissue sample is removably received into a receiving chamber.

Further features, advantages, and areas of applicability will become apparent from the description provided herein. It should be understood that the description and specific examples are intended for purposes of illustration only and are not intended to limit the scope of the present disclosure.

### DRAWINGS

The drawings described herein are for illustration purposes only and are not intended to limit the scope of the present disclosure in any way. The components in the figures are not necessarily to scale, with emphasis instead being placed upon illustrating the principles of the disclosed embodiments. In the drawings:

FIG. 1 is a side view of an illustrative system for extracting a tissue sample;

FIG. 2A is a perspective view of a rotatable sampling element of the system of FIG. 1;

FIG. 2B is a cross-sectional view of the rotatable sampling element of FIG. 2A;

FIG. 3A is a side view of an actuator handle of the system of FIG. 1;

FIG. 3B is a top view of the actuator handle of FIG. 3B;

FIG. 4 is a cross-sectional view of the actuator handle of FIGS. 3A and 3B;

FIGS. 5A, 6A, 7A, 8A, and 9A are side views of the actuator handle being manipulated to use an embodiment of a rotatable sampling element to extract a tissue sample;

FIGS. 5B, 6B, 7B, 8B, and 9B are schematic views of the rotatable sampling element operating in response to the manipulations of the actuator handle of FIGS. 5A, 6A, 7A, 8A, and 9A, respectively;

FIG. 10A is a side view of the actuator handle with the application of a vacuum source to secure and/or withdraw the tissue sample;

FIG. 10B is a schematic view of the rotatable sampling element operating in response to the application of the vacuum source of FIG. 10A to secure and/or withdraw the tissue sample;

FIGS. 11A, 12A, 13A, and 14A are side views of an embodiment of an actuator handle that receives a stylet for guiding the rotatable sampling element and/or expelling a sample;

FIGS. 11B, 12B, 13B, and 14B are schematic views of the rotatable sampling element and a distal end of the stylet operating in response to the manipulations of the actuator handle and a proximal end of the stylet of FIGS. 11A, 12A, 13A, and 14A, respectively;

FIG. 15 is a side view in partial cutaway of a rotatable sampling element including opposing cutting elements;

FIG. 16 is a cross-sectional view of an actuator handle configured to engage opposing drive shafts couplable to the opposing cutting elements of the rotatable sampling element of FIG. 15;

FIGS. 17 and 18 are illustrative counter-rotating structures useable to counter-rotate drive shafts coupled to opposing cutting elements of a counter-rotatable sampling element;

FIGS. 19A, 20A, and 21A are side views of distal ends of elongated cutting devices that are insertable through a lumen in the flexible drive shaft supporting the rotatable sampling element;

FIGS. 19B, 20B, and 21B are perspective views of the distal ends of the elongated cutting devices of FIGS. 19A, 20A, and 21A, respectively;

FIGS. 19C, 20C, and 21C are end views of the distal ends of the elongated cutting devices of FIGS. 19A, 20A, and 21A, respectively;

FIGS. 22A, 23A, 24A, and 25A are side views of an embodiment of an actuator handle into which the elongated cutting device is inserted through a lumen in a flexible drive shaft extending from the actuator handle;

FIGS. 22B, 23B, 24B, and 25B are schematic views of the elongated cutting device cutting an opening in a tissue mass and material being inserted in the opening in response to the insertion of the elongated cutting device into the actuator handle of FIGS. 22A, 23A, 24A, and 25A, respectively;

FIG. 26 is a flow diagram of an illustrative method of using a rotatable sampling element to extract a tissue sample from within a body;

FIG. 27 is a flow diagram of an illustrative method of using a rotatable sampling element guided by a stylet to extract a tissue sample from within a body; and

FIG. 28 is a flow diagram of an illustrative method of inserting an elongated cutting device through a lumen defined by a flexible shaft to cut an opening in a tissue mass and inserting a material into the tissue mass via the lumen.

### DETAILED DESCRIPTION

The following description is merely illustrative in nature and is not intended to limit the present disclosure, application, or uses. It will be noted that the first digit of three-digit reference numbers and the first two digits of four-digit reference numbers correspond to the first digit of one-digit figure numbers and the first two digits of two-digit figure numbers, respectively, in which the element first appears.

The following description explains, by way of illustration only and not of limitation, various embodiments of apparatuses, systems, and methods for extracting a tissue sample from within a body. Given by way of non-limiting overview, in various embodiments a rotatable sampling element, coupled to a flexible drive shaft, is inserted into a body and positioned by a tissue mass from which a tissue sample is to be taken. In various embodiments, the drive shaft is coupled to an actuator handle that controls the rotation of the rotatable sampling element and/or positioning of the rotatable sampling element relative to the tissue mass. The rotatable sampling element is rotated and pressed against the tissue mass to cut the tissue sample from the tissue mass.

The rotatable sampling element may include one sampling element for cutting a tissue sample or two sampling elements that may be counter rotated to cut a tissue sample. The actuator handle may provide for counter-rotation of two sampling elements. In various embodiments, the rotatable sampling element includes a cylindrical body that defines a receiving chamber to receive the tissue sample.

In various embodiments, a vacuum source may be couplable to the rotatable sampling element via the flexible drive shaft and/or the actuator handle to apply suction to the rotatable sampling element to facilitate retrieval of the tissue sample. Additionally, in various embodiments, a stylet may be insertable through the actuator handle, drive shaft, and rotatable sampling element to guide the rotatable cutting element to a desired location. It will be appreciated that various embodiments of rotatable sampling elements and other features described herein may help to facilitate the cutting of a tissue sample from a tissue mass and retrieval of the tissue sample.

Now that a non-limiting overview has been given, details will be provided by way of examples given by way of illustration only and not of limitation.

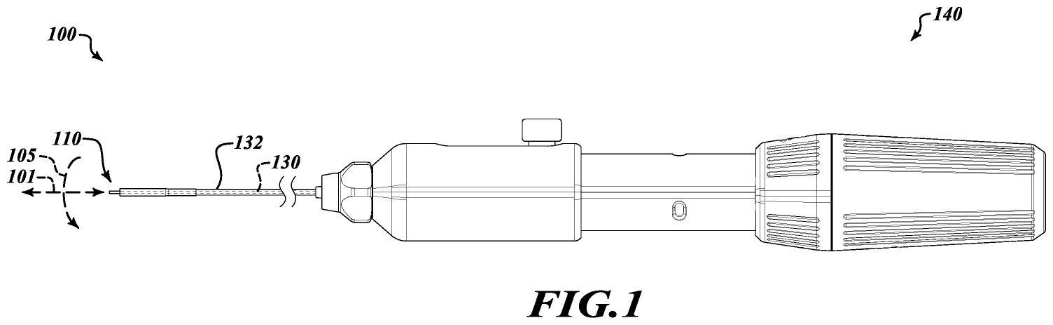

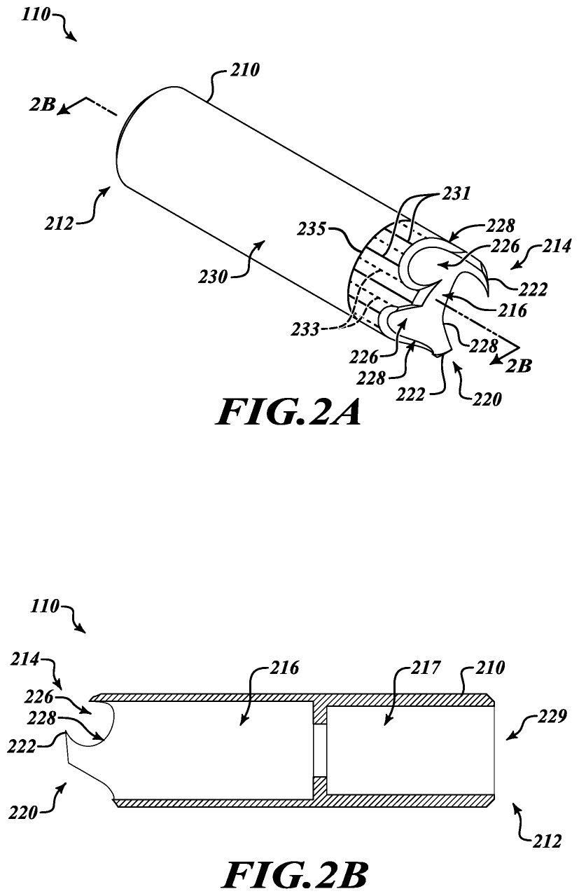

Referring to FIG. 1, a system 100 is provided for obtaining a tissue sample by using a rotatable sampling element 110 coupled to a flexible drive shaft 130 and motivated by an actuator handle 140. The rotatable sampling element 110, which is further described below with reference to FIGS. 2A and 2B, is advanced along an axis 101 to reach a tissue mass (not shown in FIG. 1) from which the sample is to be extracted. The rotatable sampling element 110 is rotatable through a curve 105 around the axis 101 as the rotatable sampling element 110 is pressed against the tissue mass to effect cutting of the tissue. The rotatable sampling element 110 is couplable with or mounted to the drive shaft 130.

The drive shaft 130 is desirably flexible to be inserted into a body where the drive shaft 130 may be maneuvered around other bodily structures (not shown in FIG. 1) to reach the tissue mass to be sampled. In various embodiments, the drive shaft 130 is shrouded within a sheath 132. The drive shaft 130 and its sheath 132 may be inserted into the body using an apparatus, such as an endoscope or bronchoscope that is configured to convey an insertion tube into a desired target region within the body. The actuator handle 140 engages the drive shaft 130 to manipulate the drive shaft 130 along the axis 101. The actuator handle 140 also is configured to motivate the drive shaft 130 to rotate through the curve 105 around the axis 101 to rotate the rotatable sampling element 110. In various embodiments, the drive shaft 130 may define a lumen (not shown in FIG. 1) that extends through to the rotatable sampling element 110 to enable suction to be applied to an interior of the rotatable sampling element 110 and/or to enable a stylet to be extended through the drive shaft 130 into and/or through the rotatable sampling element 110, as further described below. The configuration of the rotatable sampling element 110, the drive shaft 130, the actuator handle 140, other configurations, and examples of the use thereof, are described below.

Referring to FIGS. 2A and 2B, in various embodiments an illustrative rotatable sampling element 110 includes a cylindrical body 210. The cylindrical body 210 defines a receiving chamber 216 into which a tissue sample may be received upon being detached from a tissue mass, as further described below. A proximal end 212 of the rotatable sampling element 110 is configured to be couplable with the drive shaft 130 (FIG. 1). The proximal end 212 may define an opening 229 (FIG. 2B). The opening 229 may be configured to connect the cylindrical body 210 to the drive shaft 130 (FIG. 1). In various embodiments, the flexible shaft 130 may be partially received into a socket 217 adjacent the proximal end 212 of the cylindrical body 210. Additionally, the opening 229 may be fluidly coupled with a lumen (not shown in FIGS. 2A and 2B) defined by the drive shaft 130. As further described below, the lumen enables a stylet (not shown in FIGS. 2A and 2B) that extends through the drive shaft 130 to be extended into and/or through the receiving chamber 216. As also further described below, the lumen also may enable a vacuum source (not shown in FIGS. 2A and 2B) to be fluidly coupled with the receiving chamber 216 to help secure or extract the tissue sample.

Referring again to FIGS. 2A and 2B, the distal end 214 of the cylindrical body 210 of rotatable sampling element 110 defines and/or supports a cutting element 220. The cutting element 220 is configured to rotatably cut the tissue sample from a tissue mass (neither of which are shown in FIGS. 2A and 2B). The cutting element 220 includes one or more cutting surfaces to rotatably cut the tissue sample from the tissue mass. The cutting surfaces may be symmetrically arranged around a periphery of the cylindrical body 210 at the distal end 214. The cutting element 220 may include one or more ends 222. The cutting element 220 also may include one or more lateral cutting edges 228 that angle away from each of the ends 222. The ends 222 and/or the lateral cutting edges 228 are configured to slice into the tissue mass as the rotatable sampling element 110 is rotated against the tissue mass.

Each of the lateral cutting edges 228 may extend along a slot 226 defined by the cylindrical body 210 toward the distal end 214. The lateral cutting edges 228 are configured to further cut into the tissue mass as the distal end 214 of the rotatable sampling element 110 is further extended into the tissue mass after the one or more piercing ends 222 and/or lateral cutting edges 228 have cut into the tissue mass. As further described below, the rotatable sampling element 110 may include, for example, two cutting elements, where at least one of the cutting elements is counter-rotated relative to the other cutting element. In such a configuration, the lateral cutting edges in one of the cutting elements may be configured to engage lateral cutting edges of the other cutting element to scissor tissue between the lateral cutting edges.

In various embodiments, the rotatable sampling element 110 may be used under ultrasound visualization so that medical personnel may monitor the position of the rotatable sampling element 110 relative to a tissue mass and/or a lesion to be sampled. Accordingly, to enhance the visibility of the rotatable sampling element 110, an outer surface 230 of the rotatable sampling element 110 may be marked with a number of cuts 231 and/or pits 233 to reflect signal energy. The cuts 231 and/or pits 233 may be formed adjacent the distal end 214 because it is particularly desirable to be able to monitor that portion of the rotatable sampling element 110. A lateral marking 235 may be formed at a particular position to provide a visual reference point of the position of the rotatable sampling element 110. The cuts 231, pits 233, and/or lateral marking 235 may be formed by laser etching or any other process that can score or pit the surface 230 of the rotatable sampling element 110.

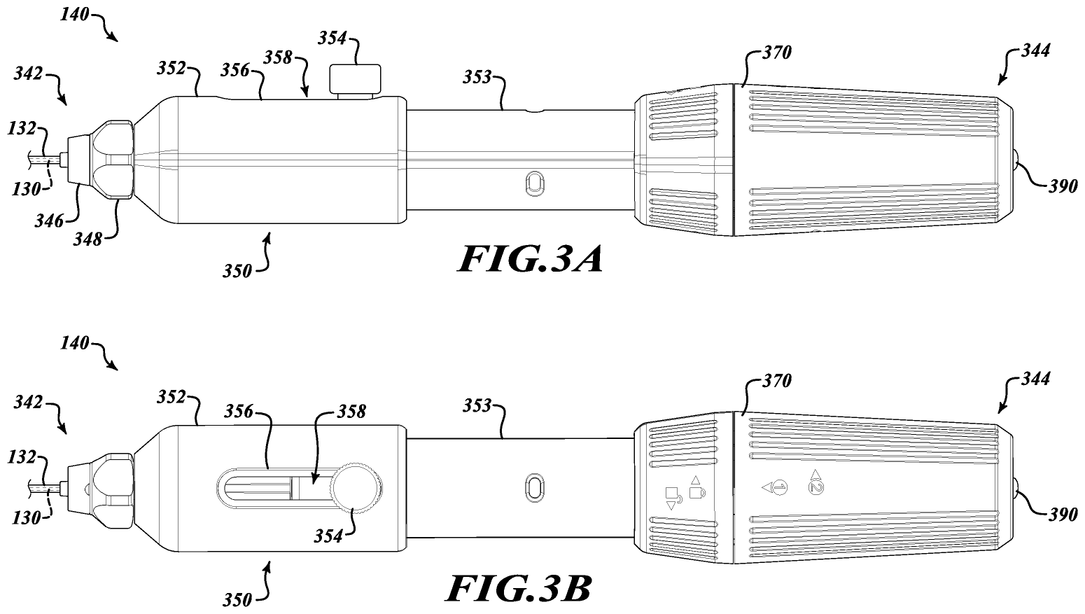

Referring to FIGS. 3A and 3B, an actuator handle 140 receives the drive shaft 130 and the sheath 132 at a distal end 342 of the actuator handle 140. In various embodiments, the actuator handle 140 causes the drive shaft 130 and the attached rotatable sampling element 110 (FIG. 1) to move along the axis 101 (FIG. 1) and be rotated along the curve 103 (FIG. 1) around the axis 101 in order to cut the tissue sample from the tissue mass.

As previously described with reference to FIG. 1, the rotatable sampling element 110 and the drive shaft 130 may be conveyed to the tissue mass (not shown in FIGS. 3A and 3B) using an endoscope, bronchoscope, or another insertion device (also not shown in FIGS. 3A and 3B) through which an elongated instrument can be extended into a body. To this end, the distal end 342 of the actuator handle 140 may include a device coupling 346, such as a threaded coupling configured to engage a mated threaded coupling on the electrosurgical device. The device coupling 346 may be rotatable to engage the mated threaded coupling on the insertion device, and may include knurled ring 348 or other control surface to facilitate rotating the device coupling 346 in order to secure the device coupling 346 to the electrosurgical device.

In various embodiments, the actuator handle 140 includes a sheath actuator 350. The sheath actuator 350 enables the sheath 132 to be moved relative to the insertion device (not shown in FIGS. 3A and 3B) to position the sheath 132, as well as the drive shaft 130 enclosed therein, relative to a region from which a sample is to be drawn. The sheath actuator 350 (FIGS. 3A and 3B) includes a slidable mechanism that includes a sleeve 352 that slidably receives a housing 353 that is mechanically engaged with the sheath 132. Movement of the housing 353 into the sleeve 352 causes the sheath 132 and the enclosed drive shaft 132 to advance within the insertion device.

To control movement of the sheath actuator 350, the sleeve 352 includes a locking device 354 which, in various embodiments, includes a knurled locking screw. The locking device 354 extends through a channel 358 in the sleeve 352 and is threadably received by the housing 353. When the locking device 354 is tightened, the locking device 354 engages mechanically and/or frictionably engages one or more sides 356 of the channel 358, holding the housing 353 in place relative to the sleeve 353. When the locking device 354 is loosened, such as by rotating the locking device 354, the locking device 354 is released from the one or more sides 356 of the channel 358. With the locking device 354 released from the sides 356 of the channel 358, the housing 353 is able to slide relative to the sleeve 352. Movement of the housing 353 relative to the sleeve 352 and the attached device coupling 346 enables the sheath 132 to be advanced toward or retracted from a tissue mass to be sampled. Positioning of the sheath is further described below with reference to FIGS. 5A-6B and FIGS. 9A and 9B.

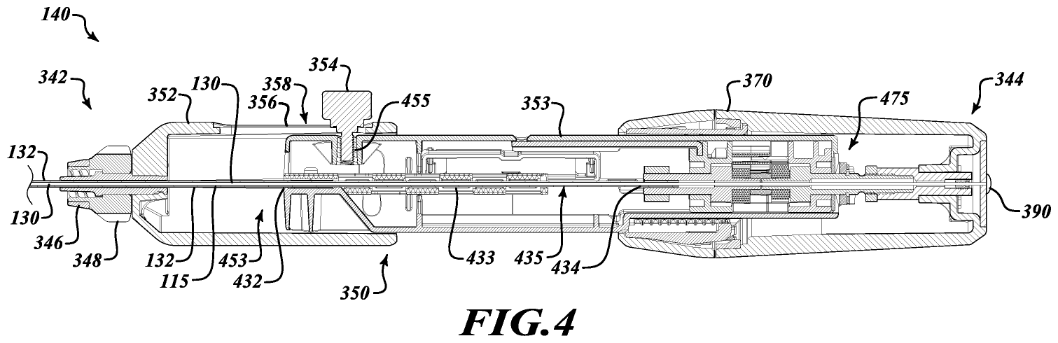

Once the sheath actuator 350 is used to position the sheath 132 at a desired position, a control actuator 370 is used to advance and rotate the drive shaft 130 to advance and rotate the rotatable sampling element 110. The control actuator 370 is mechanically coupled to the drive shaft 130 as further described with reference to FIG. 4. The control actuator 370 is movably coupled to the housing 353. Thus, once the housing 353 is secured in place relative to the sleeve 352 by the locking device 354, the control actuator 370 may be rotated and advanced over the drive shaft 353. Advancing and rotating the control actuator 370 relative to the housing 353 advances and rotates the drive shaft 130 and, in turn, the rotatable sampling element 110. Advancing the rotatable sampling element 110 causes cutting element 220 (FIGS. 2A and 2B) to cut a tissue sample from the tissue mass.

In various embodiments, a proximal end 344 of the control actuator 370 also may include a port 390 that is fluidly coupled to a lumen (not shown in FIG. 3A or 3B) defined by the drive shaft 130. As further described below, the port 390 may be configured to receive a stylet (not shown in FIGS. 3A and 3B) that may be used to guide the rotatable sampling element 110. The port 390 may be presented as a vacuum port fluidly coupled with the lumen and configured to be fluidly coupled with a vacuum source (not shown in FIGS. 3A and 3B) enable suction to be applied to the lumen to secure and/or extract the tissue sample. The port 390 may include a pierceable and/or self-sealing membrane to receive a stylet therethrough or to fluidly engage the vacuum source.

Referring to FIG. 4, the sleeve 352 defines an annular channel 453 to slidably receive the housing 353 when it is moved along the axis (FIG. 1) to advance the sheath 132 and the enclosed drive shaft 130. A threaded recess 455 in the housing 353 threadedly receives the locking device 354 to selectively secure the housing 353 relative to the sleeve 352 as previously described. As previously stated, the housing 353 is mechanically coupled to the sheath 132 at a sheath coupling 432 so that movement of the housing 353 within the sleeve 352 advances the sheath 132 into the insertion device.

Within the housing 353 rearward of the sheath coupling 432, the drive shaft 130 extends out of the sheath 132 to a shaft coupling 434 that is mechanically engaged with the control actuator 370. As the drive shaft 130 extends out of the sheath 132, a shaft support 433 sized to internally receive the drive shaft 130 provides lateral support to the shaft. Thus, as the drive shaft 130 is advanced as described further below, lateral support from the shaft support 433 may prevents the shaft from buckling.

At the shaft coupling 432, the drive shaft 130 is coupled to a rotating mechanism 475 within the control actuator 370. Rotation of the control actuator 370 causes the rotating mechanism 475 to rotate the drive shaft 130 and, in turn, causes the rotatable sampling element 110 to rotate. As further described below, in various embodiments in which there are more than one sampling element, the rotating mechanism 475 causes at least one of the rotatable sampling elements to counter-rotate relative to the other to scissor tissue, as previously described with reference to FIGS. 2A and 2B.

To advance the drive shaft 130 and, in turn, the rotatable sampling element 110, the control actuator 370 is movable longitudinally relative to the housing 353. The control actuator 370 may be slidable over the housing 353 or the control actuator 370 may be threadably coupled to the housing 353 so that rotation of the control actuator 370 causes the control actuator 370 and the associated rotating mechanism 475 to advance the rotatable sampling element 110 at the same time that the rotatable sampling element 110 is rotated. When the control actuator 370 is threadably mounted to the housing 353 or otherwise longitudinally coupled with the housing 353, sliding of the control actuator 370 may be used to advance the housing 353 into the sleeve 352 to advance the sheath, as previously described.

Continuing to refer to FIG. 4, the drive shaft 130 defines a lumen 435 that extends throughout the length of the drive shaft 130. In various embodiments, the lumen 435 is fluidly engaged with the port 390 at the proximal end 344 of the control actuator 370. The lumen 435 is configured to receive a stylet and/or a vacuum source via the port 390 as previously mentioned. As further described below, the lumen 435 allows a stylet to be extended through the drive shaft 130 and through the rotatable sampling element 110 to guide the rotatable sampling element 130. The lumen 435 also allows a vacuum source to apply suction to the rotatable sampling element 110 to aid in securing and/or extracting a tissue sample cut from a tissue mass.

Referring to FIGS. 5A and 5B, in corresponding views of the actuator handle 140, the rotatable sampling element 110, and the conjoining drive shaft 130, the components are positioned prior to advancing of the sheath 132 adjacent to a tissue mass 501 from which a sample is to be taken. In the examples shown in FIGS. 5A-13B, the tissue mass 501 includes a lesion 503 or another object from which a sample is desired. The rotatable sampling element 110, the drive shaft 130, and the sheath 132 may have been conveyed to the position shown in FIGS. 5A and 5B by an insertion tube of an insertion device (not shown), such as an endoscope or bronchoscope.

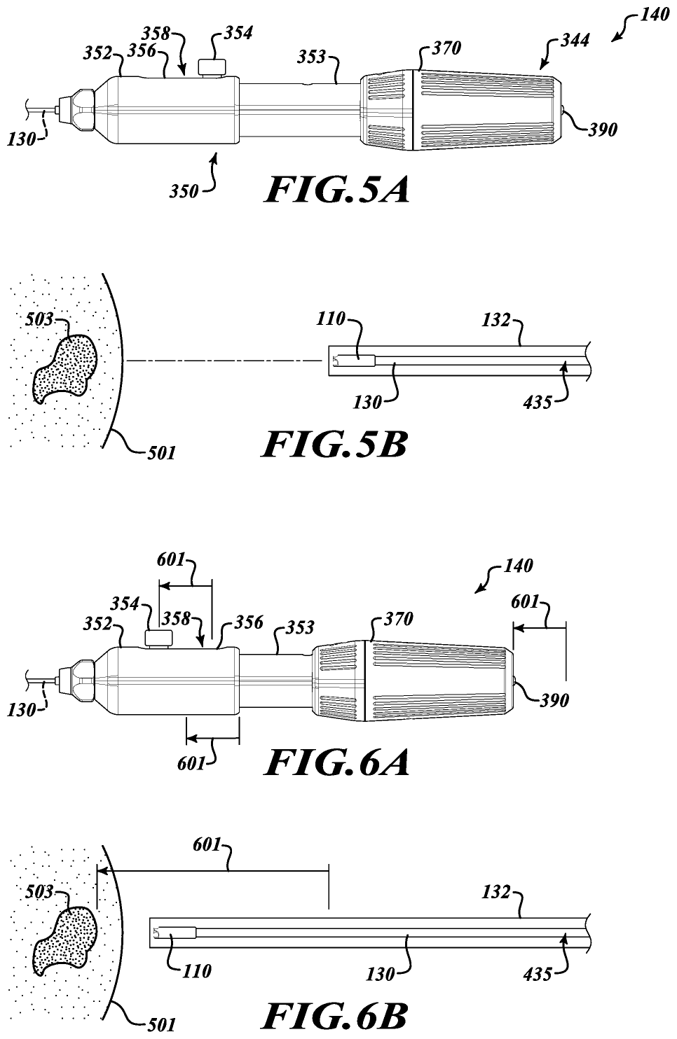

Referring to FIGS. 6A and 6B, the actuator handle 140 is manipulated to advance the sheath 132 to move the rotatable sampling element 110 to the desired sampling location adjacent the tissue mass 501. As previously described with reference to FIGS. 3A, 3B, and 4, the locking device 354 is manipulated to permit advancement of the sheath 132. Specifically, the locking device 354 extending from the housing 353 may be loosened to disengage the locking device 354 from the one or more sides 356 of the channel 358, allowing the housing 353 to slide within the sleeve 352. Sliding the housing 353 within the sleeve 352 by a distance 601 advances the sheath 132, the drive shaft 130, and the rotatable sampling element 130 through a corresponding distance toward the tissue mass 501. Once in place, the locking device 354 is secured to hold the sheath 132 in place to prepare for advancement of the drive shaft 130 and the rotatable cutting element 110.

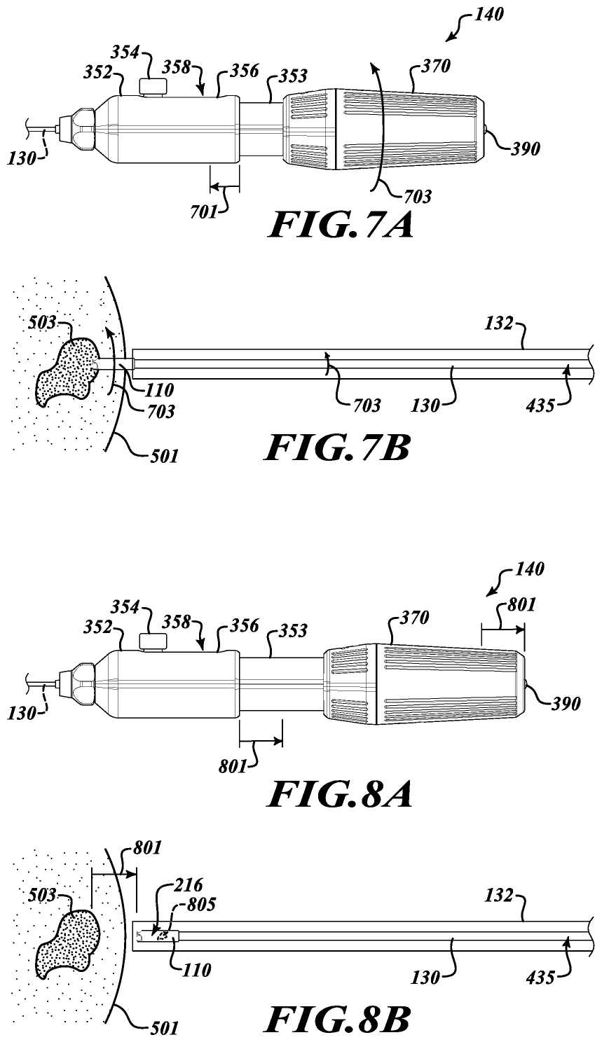

Referring to FIGS. 7A and 7B, with the sheath 132 having been advanced to a position adjacent to the tissue mass 501, the rotatable sampling element 110 is manipulated to excise a sample. In various embodiments in which the control actuator 370 is threadably coupled with the housing 353, a user (not shown) may rotate the control actuator 370 in a direction 703 to rotate the drive shaft 130 and the rotatable sampling element 110 in the same direction. At the same time, the rotatable sampling element 110 is advanced so as to press the rotatable sampling element 110 against and into the tissue mass 501 and the lesion 503. The advancement and rotation of the rotatable sampling element 110 cuts a sample of the lesion 503 and/or the tissue mass 501 as a result of the movement of the ends 222 and/or the lateral cutting edges 228 (FIGS. 2A and 2B) of the rotatable cutting element 110. As the sample is separated from the lesion 503 and/or the tissue mass 501, the sample is received into the receiving chamber 216 (FIGS. 2A and 2B) of the rotatable sampling element 110, as further described below.

As previously described, in various embodiments, the control actuator 370 is threadably mounted to the housing so that rotation of the control actuator 370 both rotates and advances the drive shaft 130 and the rotatable sampling element 110. In other embodiments, the control actuator 370 may be separately slidable and rotatable relative to the housing 353 such that a user rotates and slides the control actuator 370 to rotate and advance the drive shaft 130 and the rotatable sampling element 110 as previously described.

Referring to FIGS. 8A and 8B, once a sample 805 has been excised from the lesion 503 and/or the tissue mass 501 and received into the receiving chamber 216, the rotatable sampling element 110 may be withdrawn from the tissue mass 501. The rotatable sampling element 110 is retracted from the tissue mass 501 by a distance 801 by withdrawing the control actuator 370 by a corresponding distance 801. For example, when the control actuator 370 is threadably coupled with the housing 353, the control actuator 370 may be withdrawn by rotating the control actuator 370 in a direction opposite to that used to extend the drive shaft 130 and the rotatable sampling element 110 as described with reference to FIGS. 7A and 7B. Alternatively, the control actuator 370 may be slid in an opposing direction along the housing 353 or otherwise moved to retract the drive shaft 130 and the rotatable sampling element 110. As shown in FIG. 8B, the drive shaft 130 desirably may be withdrawn to cause the rotatable sampling element 110 to be retracted within the sheath 132. By retracting the rotatable sampling element 110 within the sheath 132, the sheath 132 prevents the rotatable sampling element 110 from impacting the insertion device (not shown) used to insert the sheath 132, drive shaft 130, and rotatable sampling element 110 into the body, thereby preventing damage to either the rotatable sampling element 110 or the insertion device.

Referring to FIGS. 9A and 9B, once the sample 805 is procured and the drive shaft 130 and rotatable sampling element 110 are withdrawn from the tissue map, the sheath 132 and the enclosed drive shaft 130 and rotatable sampling element 110 may collectively be withdrawn from the tissue mass 501. The withdrawal process is comparable to that used to extend the sheath 132 as previously described with reference to FIGS. 6A and 6B. The locking device 354 may be loosened to disengage the locking device 354 from the one or more sides 356 of the channel 358, allowing the housing 353 to slide within the sleeve 352. Sliding the housing 353 out of the sleeve 352 by a distance 901 withdraws the sheath 132, the drive shaft 130, and the rotatable sampling element 130 through a corresponding distance 901 away from the tissue mass 501. Once the sheath is withdrawn, the locking device 354 may be tightened to secure the sheath 132 in place in preparation for removal from the body and/or the insertion device (not shown).

In various embodiments, the tissue sample 805 cut from the lesion 503 and/or tissue mass 501 received within the receiving chamber 216 of the rotatable cutting element 110 may be frictionally held within the receiving chamber 216. Alternatively, suction may be used to secure the tissue sample 805 and/or to at least partially withdraw the tissue sample 805 into the receiving chamber 216 of the rotatable sampling element 110 and/or into the drive shaft 130.

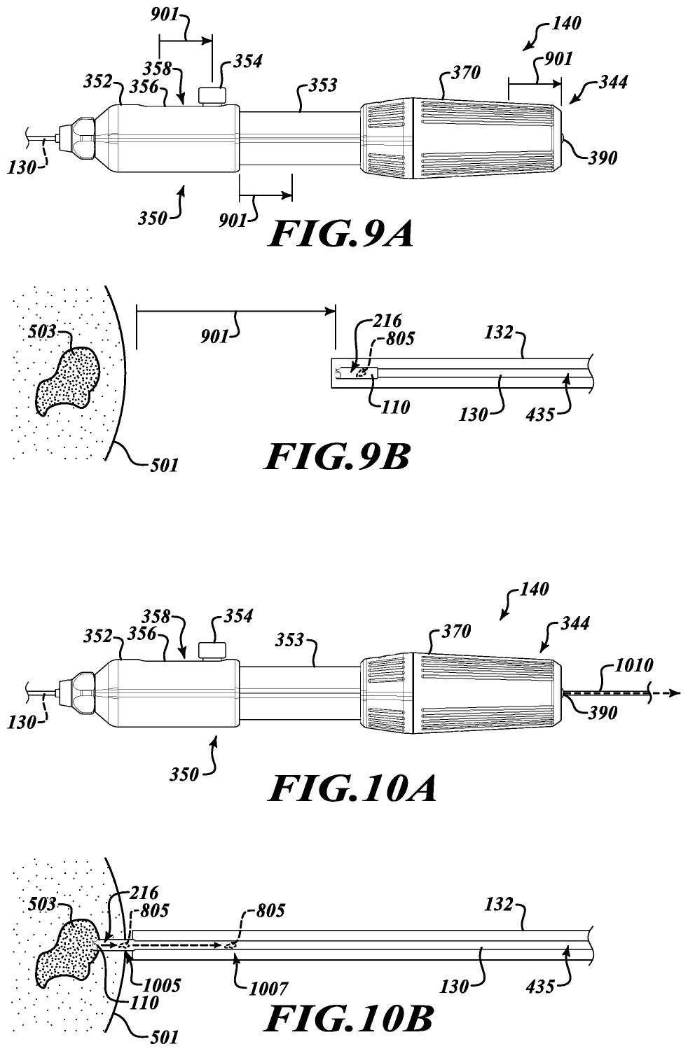

Referring to FIGS. 10A and 10B, a vacuum source 1010 may be coupled to the port 390 on the actuator handle 140. The vacuum source 1010 may be a mechanical device, such as a syringe or other handpump, or the vacuum source 1010 may include an electrically-powered pump. As previously described, the port 390 is fluidly coupled with the lumen 435 defined by the drive shaft 130. As a result, coupling the vacuum source 1010 to the port 390 and applying suction to the port 390 applies suction to the lumen 435. The application of suction to the port 390 thus may draw or secure the tissue sample 805 into the receiving chamber 216 of the rotatable sampling element 110 at a location 1005 or the sample 805 may be drawn into the lumen 435 defined by the drive shaft 130 at a location 1007. After the rotatable sampling element 110 is removed from the body, the sample 805 may be dislodged for collection and evaluation. The sample 805 may be mechanically dislodged from the receiving chamber 216 with an implement or by air pressure applied to the port 390 and through the lumen 435 to expel the sample 805.

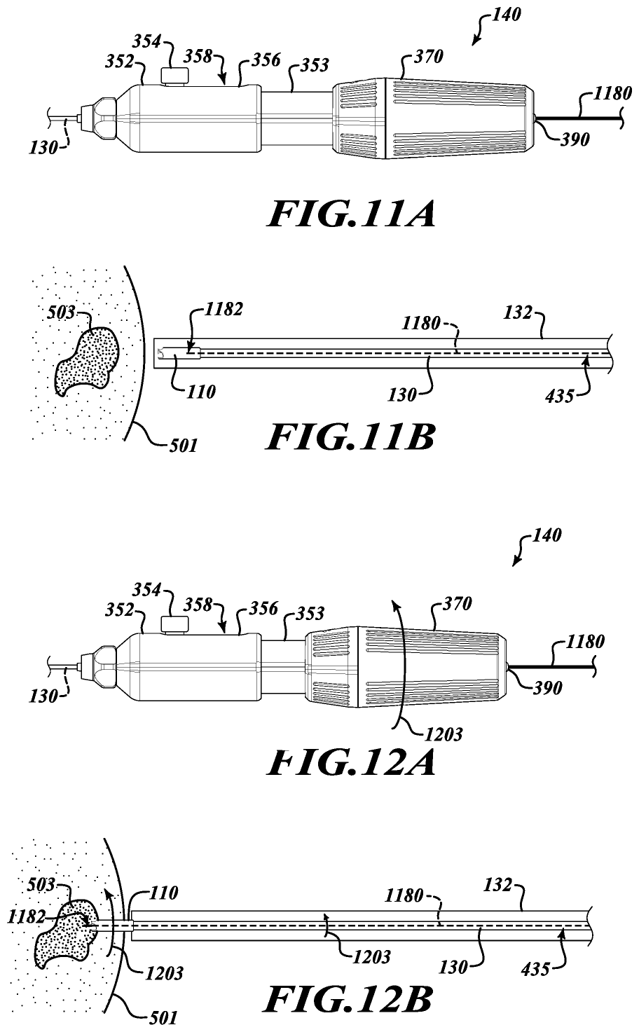

Referring to FIGS. 11A through 14B, a stylet 1180 may also be used in order to guide the rotatable sampling element 110 and/or to expel a sample from the apparatus. The stylet 1180 may include a rigid but flexible wire sized to be slidably passed through the lumen 435 defined by the drive shaft 130. Referring to FIGS. 10A and 10B, the stylet 1180 may be inserted into the lumen 435 via the port 390 at the proximal end 344 of the control actuator 370 and fed through the lumen 435 until a distal end 1182 of the stylet 1180 reaches the rotatable sampling element 110. The stylet 1080 may be inserted through the drive shaft 130 before or after the rotatable sampling element 1010 and drive shaft 130 are inserted into the body.

Referring to FIGS. 11A and 11B, the stylet 1180 may be further extended by pushing the stylet 1180 into the port 390 by a distance 1201. Further extending the stylet 1180, without advancing the drive shaft 130 or the rotatable sampling element 110, can cause the distal end 1182 of the stylet 1080 to extend beyond a distal end 1202 of the rotatable sampling element 110. Optionally guided by imaging technologies, such as ultrasound or other technologies, the distal end 1182 of the stylet 1180 may be extended to or into a point of interest, such as the lesion 503, within the tissue mass 501. The distal end 1182 of the stylet 1180 may be inserted into the lesion 503 or other point of interest to anchor the distal end 1182 of the stylet 1180 to provide an internal guide wire to direct extension of the rotatable sampling element 110 and the drive shaft 130.

Continuing to refer to FIGS. 12A and 12B, with the stylet 1180 guiding the apparatus to the lesion 503, the actuator handle 370 is manipulated as described with reference to FIGS. 7A and 7B to advance the drive shaft 130 and the rotatable sampling element 110 into the tissue mass 501 to the lesion 503. Rotating the control actuator 370 in a direction 1203, the drive shaft 130 and the rotatable sampling element 110 are rotated to cut a sample from the lesion 503 and/or the tissue mass 501.

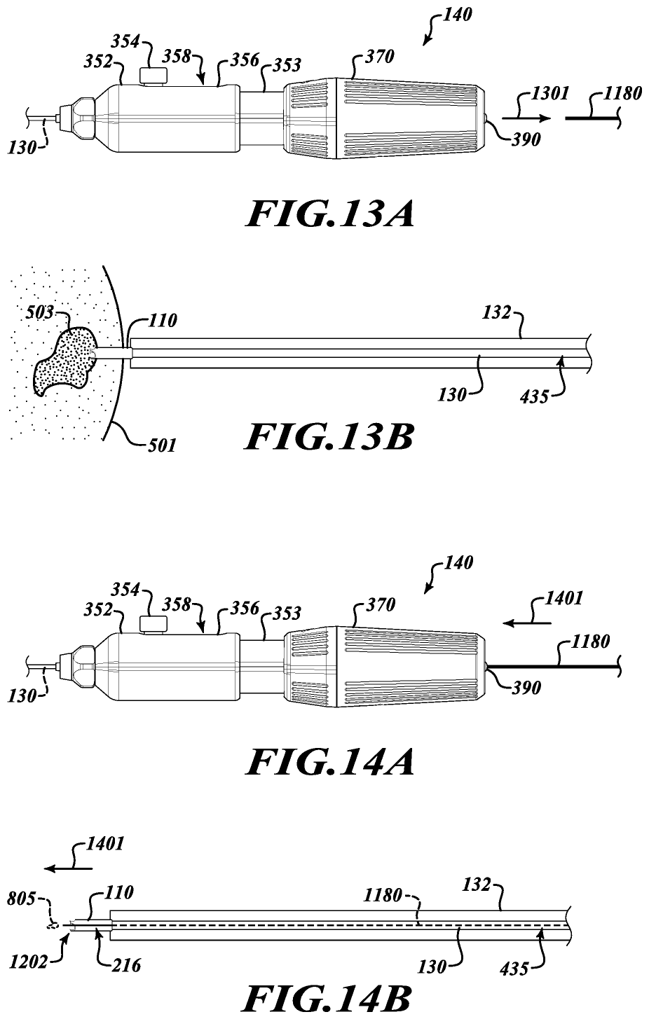

Referring to FIGS. 13A and 13B, with the rotatable sampling element 110 in place at its desired destination, the stylet 1080 may be withdrawn through the lumen 435 defined by the drive shaft 130. The stylet 1180 may be withdrawn from the port 390 in a direction 1301 to withdraw the stylet 1080 from the rotatable sampling element 110. Withdrawing the stylet 1180 may prevent the distal end 1182 of the stylet from obstructing the receiving chamber 216 so that a tissue sample may be received into the receiving chamber 216. The control actuator 370 then may be engaged to cut a tissue sample to be received within the receiving chamber 216, as previously described with reference to FIGS. 7A-8B.

The stylet 1180 may also be fully withdrawn from the lumen 435 defined by the drive shaft 130 and from the actuator handle 140 via the port 390. With the stylet 1180 withdrawn from the lumen 435 defined by the drive shaft 130, the vacuum source 1010 (FIG. 10A) may be coupled to the port 390 to secure and/or extract the tissue sample (not shown in FIGS. 13A and 13B) as previously described with reference to FIGS. 10A and 10B.

Referring to FIGS. 14A and 14B, it will be appreciated that the stylet 1180 also may be used to expel the tissue sample 805 from the receiving chamber 216 or the lumen 435. By inserting the stylet 1180 through the lumen 435 in a direction 1401, the stylet 1180 may be used to mechanically drive the tissue sample 805 from the lumen 435 and/or the receiving chamber 216 of the rotatable sampling element 110.

As previously described, in various embodiments, a rotatable sampling element may include multiple cutting elements to cut tissue. As previously mentioned, two cutting elements may be used in a configuration in the elements may be relatively counter-rotated to scissor tissue.

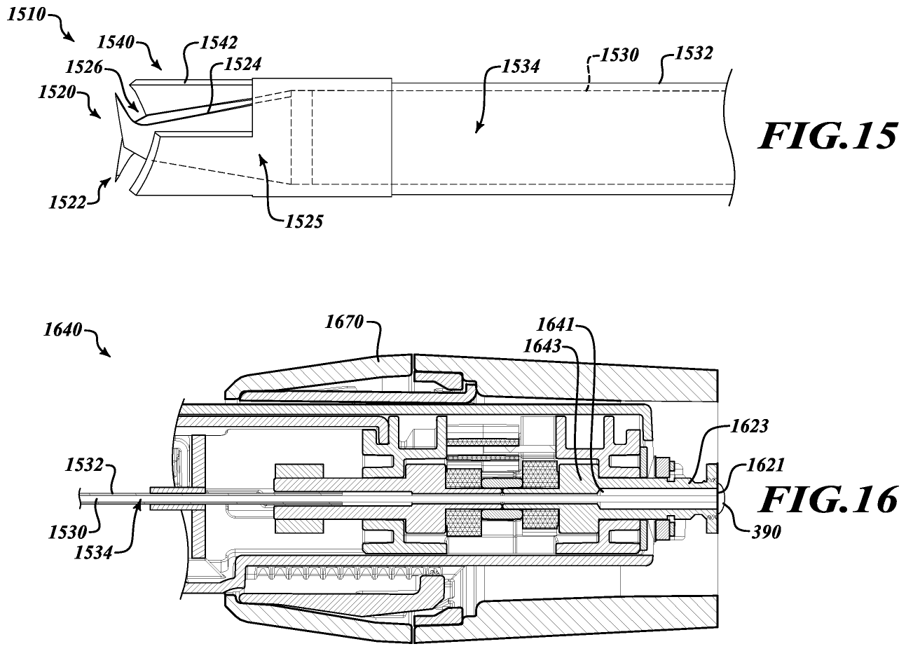

Referring to FIG. 15, a rotatable sampling member 1510 includes an inner cutting member 1520 and an outer cutting member 1540. In various embodiments, the inner cutting member 1520 may be like the rotatable sampling element 110 as previously described. Alternatively, the inner cutting member 1520 may be of a different configuration. For example, the inner cutting member 1520 may include tips 1522 and lateral cutting surfaces 1524 angled to cut, at least in part, across a rotational direction of the inner cutting member 1520. The outer cutting member 1540 includes opposing lateral cutting surfaces 1542 that face the lateral cutting surfaces 1524 of the inner cutting member 1520. As the inner cutting member 1520 is counterrotated relative to the outer cutting member 1540, the lateral cutting surfaces 1524 and 1542 approach each other across a slot 1526 and then pass across each other to scissor tissue. The inner cutting member 1520 is coupled to a flexible inner drive shaft 1530 and the outer cutting member 1540 is coupled to a flexible outer drive shaft 1532. As previously described with reference to FIGS. 4 and 10A-14B, the inner drive shaft 1530 may define a lumen 1534 that is fluidly coupled with a receiving chamber 1525 of the inner cutting member 1520. The lumen 1534 enables the use of a stylet and/or application of a vacuum source, as previously described with reference to FIGS. 10A-14B.

To facilitate scissoring of tissue by the opposing lateral cutting surfaces 1524 and 1542, the inner cutting member 1520 and the outer cutting member 1540 are counterrotated relative to each other. This relative counterrotation may be facilitated by holding one of the cutting members 1520 and 1540 in a fixed position while rotating the opposing cutting member. For example, in some embodiments the inner cutting member 1520 may be rotated while holding the outer cutting member 1540 in a fixed position to achieve a relative counterrotation of the cutting members 1520 and 1540. In some other embodiments, both cutting members 1520 and 1540 may be rotated in opposing directions to achieve counterrotation of the cutting members 1520 and 1540.

Referring to FIG. 16, the inner drive shaft 1530 and the outer drive shaft 1532 are separately engaged by structures within an actuator handle 1640. In various embodiments where the outer drive shaft 1532 remains stationary, a proximal end 1641 of the outer drive shaft 1532 is mechanically coupled with a non-rotating structure 1643 within the actuator handle 1540. At the same time, a proximal end 1621 of the inner drive shaft 1530 is coupled to a rotatable structure 1623 that is rotatable by a rotatable control actuator 1670. As a result, rotation of the rotatable control actuator 1670 relatively counterrotates the inner drive shaft 1530 and the outer drive shaft 1532 by rotating the inner drive shaft 1530 while holding the outer drive shaft 1532 in a stationary position.

In various embodiments, both the inner drive shaft 1530 and the outer drive shaft 1532 may be counter-rotated by oppositely rotating both the inner drive shaft 1530 and the outer drive shaft 1532. Thus, instead of the outer drive shaft 1532 being mechanically coupled with a non-rotating structure 1643, as previously described with reference to FIG. 15, the outer drive shaft 1532 may be mechanically coupled to an oppositely rotating structure. Thus, when the control actuator 1670 is rotated, both the inner drive shaft 1530 and outer drive shaft 1532 are rotated in opposing directions.

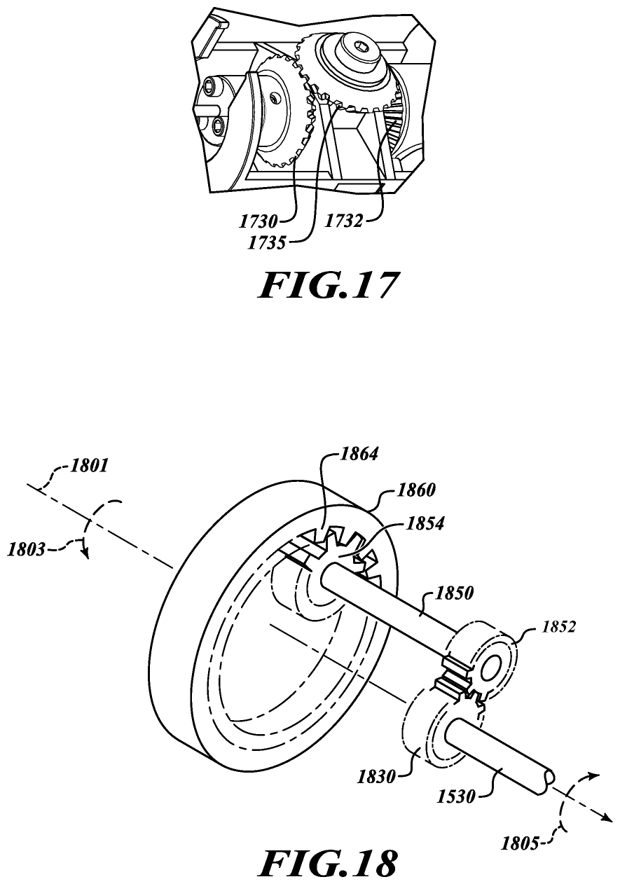

Referring to FIGS. 17 and 18, two examples of different types of mechanisms that may be used to oppositely rotate the inner drive shaft 1530 and outer drive shaft 1532 (FIGS. 15 and 16) use counter-rotating gears. Referring to FIG. 17, a first beveled gear 1730 may be mechanically couplable to the inner drive shaft 1530 and a second beveled gear 1732 may be mechanically couplable to the outer drive shaft 1532. An interconnecting beveled gear 1735 engages the first beveled gear 1730 and the second beveled gear 1732. As a result, when one of the beveled gears 1730 and 1732 is rotated, such as by rotation of a control actuator (not shown in FIG. 17), the other of the beveled gears is rotated in a opposite direction to counter-rotate the drive shafts 1530 and 1532.

Referring to FIG. 18, in another alternative embodiment, a first gear 1830 is couplable to the inner drive shaft 1530. The first gear 1830 engages a first linkage gear 1852 on a rotatable linkage 1850. A second linkage gear 1854 that extends from the rotatable linkage 1850 engages a second gear 1864 coupled with a rotating member 1860 couplable to the outer drive shaft 1532 (not shown in FIG. 18). As a result, when the rotating member 1860 is rotated, such as by rotation of a control actuator 370 (not shown in FIG. 18), the rotatable linkage 1850 causes the first gear 1830 to rotate in an opposite direction. Both of these illustrative structures, and others, may be used to counter-rotate the drive shafts by rotating each of the drive shafts in opposite directions.

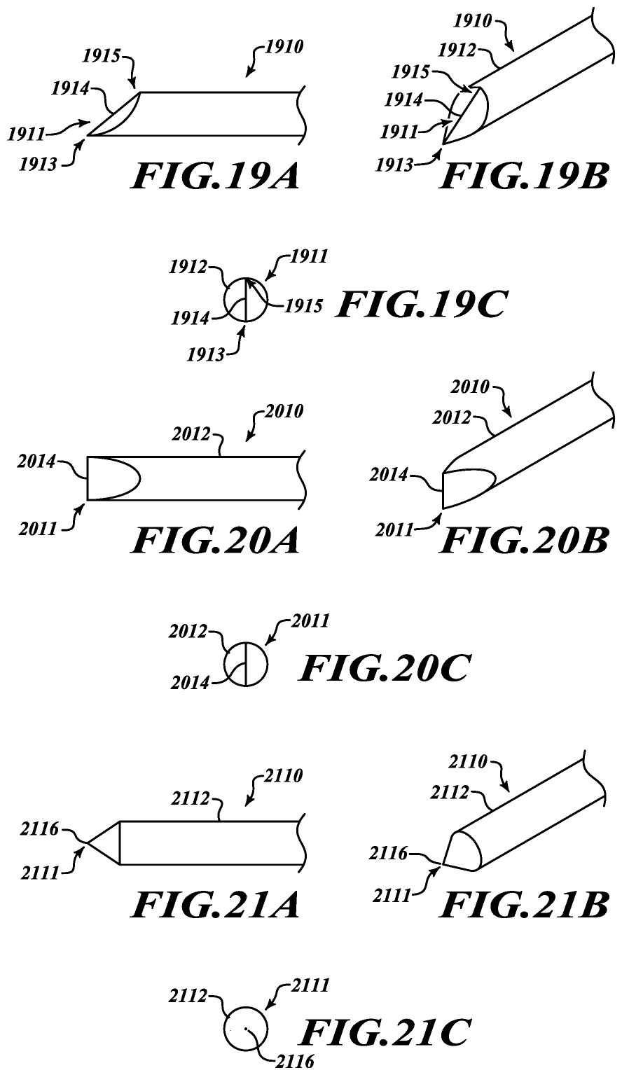

Referring to FIGS. 19A-21C, elongated cutting devices having differently shaped distal ends are insertable through the lumen 435 of the flexible drive shaft 130 (FIGS. 5A-14B). In various embodiments, an elongated cutting device is used to cut into a tissue mass and/or a lesion, such as the tissue mass 501 and the lesion 503. The elongated cutting device is pushed into the lumen 435 and fed through the lumen until the distal end reaches the tissue mass 501 where the elongated cutting device is pressed into the tissue mass 501 to cut an opening in the tissue mass 501 and/or into the lesion 503.

Referring to FIGS. 19A-19C, an elongated cutting device 1910 has a shaft 1912 that is sized to be inserted through the lumen 435 of the drive shaft 130. The shaft 1912 is flexible enough to deform to follow the course of the lumen 435 as the elongated cutting device 1910 is pushed into the lumen 435. A distal end 1911 of the elongated cutting device 1910 of FIGS. 19A-19C has an angled cutting edge 1914, angling from a leading end 1913 to a trailing end 1915. As the distal end 1911 is pressed against tissue, the leading end 1913 may pierce the tissue. Then, as the distal end 1911 is pressed into the tissue, the angled cutting edge 1914 slices the tissue as the trailing end 1915 is also advanced into the tissue.

Referring to FIGS. 20A-20C, another embodiment of an elongated cutting device 2010 has a shaft 2012 that is sized to be inserted through the lumen 435 of the drive shaft 130 and flexible enough to deform to follow the course of the lumen 435 as the elongated cutting device 2010 is pushed into the lumen 435. A distal end 2011 of the elongated cutting device 2010 of FIGS. 20A-20C has a straight cutting edge 2014 that extends transversely across a width of the shaft 2012. As the distal end 2011 is pressed against tissue, the cutting edge 2014 slices and parts the tissue as the elongated cutting device is pressed into the tissue.

Referring to FIGS. 21A-21C, another embodiment of an elongated cutting device 2110 has a shaft 2112 that is sized to be inserted through the lumen 435 of the drive shaft 130 and flexible enough to deform to follow the course of the lumen 435 as the elongated cutting device 2110 is pushed into the lumen 435. A distal end 2111 of the elongated cutting device 2110 of FIGS. 21A-21C tapers from the shaft 2112 to a sharpened point 2116. As the distal end 2111 is pressed against tissue, the sharpened point 2116 pierces the tissue. As the distal end 2111 is further advanced into the tissue, the sharpened point continues to pierce and part the tissue.

Although three embodiments of the elongated cutting devices 1910, 2010, and 2110 (FIGS. 19A-21C, respectively) are described, additional elongated cutting devices may be used to pierce, slice, and part tissue for use with the methods described below. For example, a cutting edge that tapers to a flat point or a cutting edge with orthogonal cutting edges (neither of which are shown in FIGS. 19A-21C) also may be used.

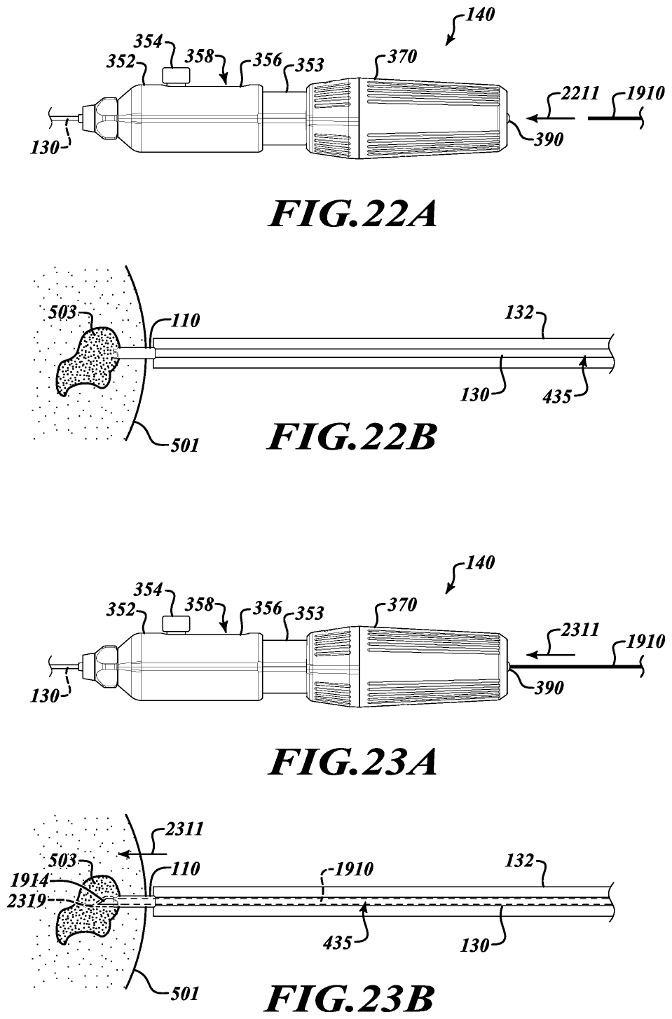

Referring to FIGS. 22A-25B, using one of the elongated cutting devices 1910, 2010, and 2110 as previously described (FIGS. 19A-21C, respectively), an opening may be cut into a tissue mass and/or lesion in order to deposit material in the opening. As further described below, once an opening is cut into a tissue mass and/or a lesion therein, materials to test the tissue, dye the tissue for imaging, treat the tissue, or for other purposes may be introduced into the opening. An actuator handle 140 may be used to advance a sheath 132 and an enclosed flexible drive shaft 130 to position the flexible shaft 130 at a desired location adjacent a tissue mass, as previously described with reference to FIGS. 5A-14B.

In various embodiments as previously described with reference to FIGS. 11A-13B, a stylet 1180 may also be used in order to guide the flexible shaft 130 to a tissue mass 501 and/or lesion 503. As previously described, The stylet 1180 may be inserted into the lumen 435 via the port 390 at the proximal end 344 of the control actuator 370 and fed through the lumen 435 until a distal end 1182 of the stylet 1180 passes out of the flexible drive shaft 130 (and through the rotatable sampling element 110) and into the tissue mass 501 and/or the lesion 503. The flexible shaft 130 then may be advanced using the control actuator 370 with the flexible shaft 130 sliding over the stylet 1180, the stylet 1180 thus guiding the flexible shaft 130 to the tissue mass 501 and/or lesion 503. The stylet 1180 then may be withdrawn from the lumen 435, as previously described with reference to FIGS. 13A and 13B.

Referring to FIGS. 23A and 23B, an elongated cutting device, such as the elongated cutting device 1910 (FIGS. 19A-19C), is moved in a direction 2211 to insert the elongated cutting device 1910 into the port 390 of the control actuator 370. As previously described, the port 390 is coupled with the lumen 435, enabling the elongated cutting device to pass into and through the lumen 435. It will be appreciated that the elongated cutting device 1910 is slidably advanced through the lumen 425 until the elongated cutting device 1910 reaches into the tissue mass 501 to the lesion 503.

Referring to FIGS. 23A and 23B, the elongated cutting device 1910 is further advanced in a direction 2311 to cause the elongated cutting device to cut an opening 2319 in the tissue mass 501 and/or the lesion 503. As previously described with reference to FIGS. 19A-19C, the elongated cutting device 1910 includes a cutting edge 1914 to pierce, cut, and separate tissue to form the opening 2319 around the distal end of the elongated cutting device 1910.

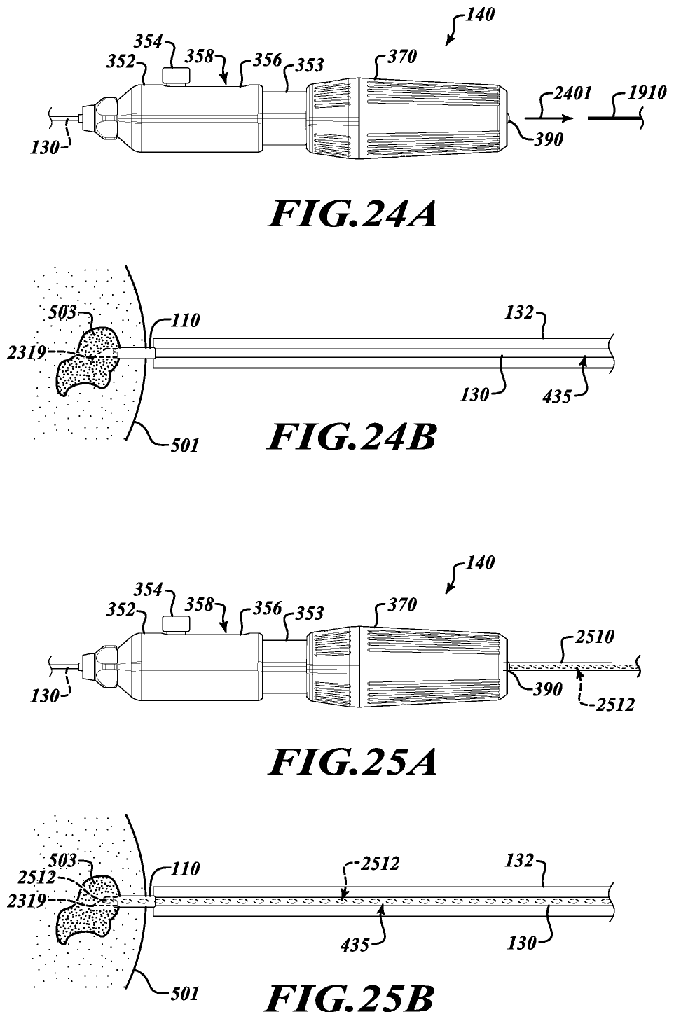

Referring to FIGS. 24A and 24B, after the elongated cutting device 1910 has been used to form the opening 2319, the elongated cutting device 1910 is withdrawn from the lumen 435 by drawing the elongated cutting device 1910 in a direction 2401. The elongated cutting device 1910 thus may be removed from the lumen 435 entirely. With the withdrawal of the elongated cutting device 1910, the lumen 435 is open from the port 390 in the control actuator 370 to the opening 2319 formed by the elongated cutting device 1910.

Referring to FIGS. 25A and 25B, a material source 2510, such as a pump, syringe, or other device, is coupled to the port 390. From the material source 2510, a material 2512, such as a testing, dying, or therapeutic agent, is fed through the port 390 into the lumen and into the opening 2319 that was formed by the elongated cutting device 2319. The material 2512 may be a liquid, gas, or a solid. In the case of a solid, the material source 2510 may have to pump the solid into the lumen 435 with a gas or liquid acting as a propellant.

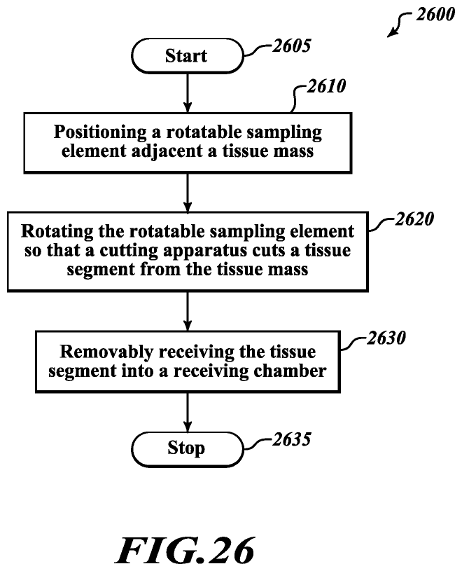

Referring to FIG. 26, an illustrative method 2600 of extracting a tissue sample is provided. The method 2600 starts at a block 2605. At a block 2610, a rotatable sampling element is positioned adjacent a tissue mass, as described with reference to FIGS. 5A-6B. At a block 2620, the rotatable sampling element is rotated so that a cutting apparatus cuts a tissue sample from the tissue mass, as described with reference to FIGS. 7A and 7B and FIGS. 12A and 12B. At a block 2630, the tissue sample is removably received into a receiving chamber, as previously described with reference to FIGS. 8A and 8B. The method 2600 ends at a block 2635.

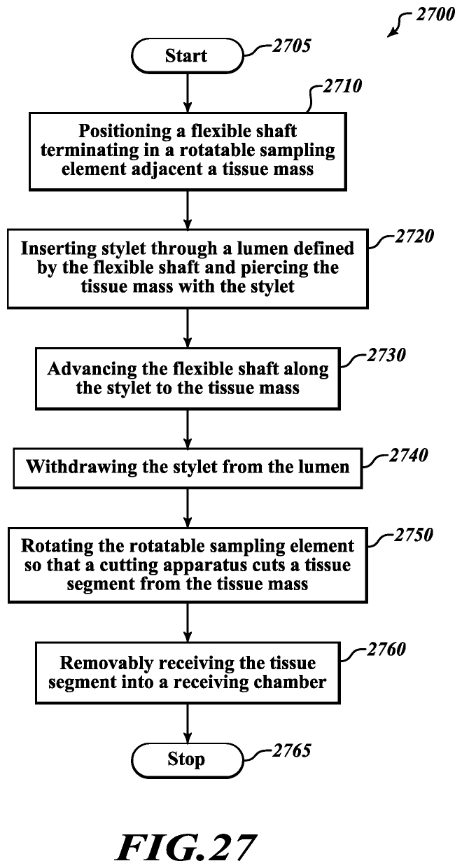

Referring to FIG. 27, an illustrative method 2700 of extracting a tissue sample including the use of a stylet is provided. The method 2700 starts at a block 2705. At a block 2710, a flexible shaft terminating in a rotatable sampling element is positioned adjacent a tissue mass, as described with reference to FIGS. 11A and 11B. At a block 2720, a stylet is inserted through a lumen defined by the flexible shaft and the stylet pierces the tissue mass. At a block 2730, the flexible shaft is moved along the stylet to the tissue mass, the stylet serving as a guide for the flexible shaft, as described with reference to FIGS. 12A and 12B. At a block 2740, the stylet is withdrawn from the lumen, as described with reference to FIGS. 13A and 13B. At a block 2750, the rotatable sampling element is rotated so that a cutting apparatus cuts a tissue sample from the tissue mass, as described with reference to FIGS. 12A and 12B. At a block 2760, the tissue sample is removably received into a receiving chamber, as previously described with reference to FIGS. 8A and 8B. The method 2700 ends at a block 2765.

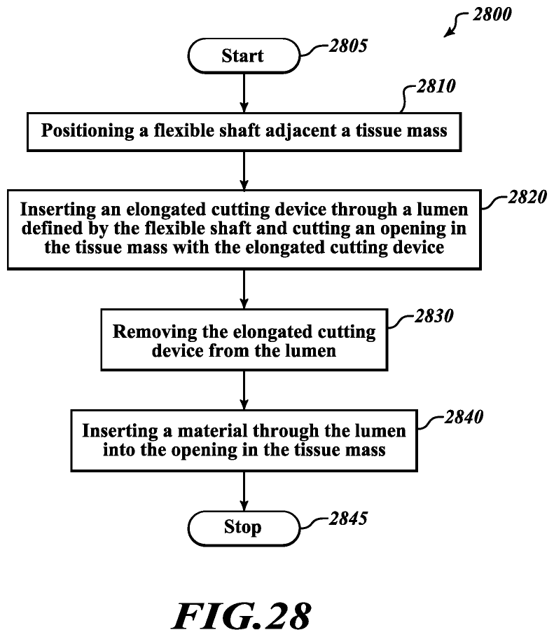

Referring to FIG. 28, an illustrative method 2800 of cutting an opening in a tissue mass and/or lesion is provided. The method 2800 starts at a block 2805. At a block 2810, a flexible shaft is positioned adjacent a tissue mass, as described with reference to FIGS. 6A and 6B and FIGS. 11A and 11B. At a block 2820, an elongated cutting device is inserted through a lumen defined by the flexible shaft and cuts an opening in the tissue mass, as described with reference to FIGS. 23A and 23B. At a block 2830, the elongated cutting device is withdrawn from the lumen, as described with reference to FIGS. 24A and 24B. At a block 2840, a material is inserted through the lumen into the opening in the tissue mass, as described with reference to FIGS. 25A and 25B. The method 2800 ends at a block 2845.

It will be appreciated that the detailed description set forth above is merely illustrative in nature and variations that do not depart from the gist and/or spirit of the claimed subject matter are intended to be within the scope of the claims. Such variations are not to be regarded as a departure from the spirit and scope of the claimed subject matter.

What is claimed is:

1. An apparatus comprising:
a rotatable sampling element including:
a cylindrical body defining a receiving chamber, the receiving chamber being configured to receive therein a tissue sample cut from a tissue mass; and
a cutting apparatus disposed at a distal end of the cylindrical body to cut the tissue sample from the tissue mass abutting the distal end responsive to rotation of the cylindrical body as the cutting apparatus is pressed against the tissue mass; and
a flexible drive shaft having a distal end fixably engaged with a proximal end of the rotatable sampling element, the flexible drive shaft being linearly movable to motivate the rotatable sampling element along an axis to press the cutting apparatus against the tissue mass and rotatable to impart rotational force to the rotatable sampling element to cause the rotatable sampling element to rotate around the axis.

2. The apparatus of claim 1, wherein the flexible drive shaft defines therein a lumen fluidly coupled with the receiving chamber.

3. The apparatus of claim 2, wherein the flexible drive shaft is configured to enable the lumen to slidably and removably receive therein a stylet extendable through the lumen to engage the tissue mass.

4. The apparatus of claim 1, wherein the cutting apparatus includes at least one cutting surface inclined relative to the axis in a first rotational direction.

5. The apparatus of claim 4, further comprising an additional rotatable sampling element including:
an additional second cylindrical body concentrically disposed around the cylindrical body and supporting at least one additional cutting apparatus at a distal end, wherein the additional cutting apparatus includes at least one additional cutting surface inclined relative to the axis and facing an opposing rotational direction opposite to the first rotational direction, the cylindrical body being rotatable relative to the additional cylindrical body to scissor the tissue mass between the cutting surface and the additional cutting surface responsive to relative counter-rotation of the cylindrical body and the additional cylindrical body; and
an additional flexible drive shaft concentrically disposed around the flexible drive shaft and fixably engaged with a proximal end of the additional rotatable sampling element, wherein the additional flexible drive shaft is laterally movable with the flexible drive shaft and rotationally independent of the flexible drive shaft to enable counter-rotation of the cylindrical body relative to the additional cylindrical body.

6. The apparatus of claim 1, further comprising a sheath configured to receive therein the rotatable sampling element and at least a portion of the flexible drive shaft and further configured to convey the rotatable sampling element adjacent the tissue mass.

7. A system comprising:
a rotatable sampling element including:
a cylindrical body defining a receiving chamber, the receiving chamber being configured to receive therein a tissue sample cut from a tissue mass; and
a cutting apparatus disposed at a distal end of the cylindrical body to cut the tissue sample from the tissue mass abutting the distal end responsive to rotation of the cylindrical body as the cutting apparatus is pressed against the tissue mass;
a flexible drive shaft having a distal end fixably engaged with a proximal end of the rotatable sampling element, the flexible drive shaft being linearly movable to motivate the rotatable sampling element along an axis to press the cutting apparatus against the tissue mass and rotatable to impart rotational force to the rotatable sampling element to cause the rotatable sampling element to rotate around the axis; and
an actuator handle including a rotatable actuator mechanically couplable with a proximal end of the flexible drive shaft to impart the rotational force to the flexible drive shaft.

8. The system of claim 7, wherein the flexible drive shaft defines therein a lumen fluidly coupled with the receiving chamber and a port defined by the actuator handle.

9. The system of claim 8, further comprising a stylet configured to be slidably and removably received by the port and the lumen to extend through the lumen to engage the tissue mass.

10. The system of claim 9, wherein the lumen is slidable along the stylet to guide the rotatable sampling element to the tissue mass.

11. The system of claim 9, wherein the port is configured to receive a vacuum source configured to apply suction to the port and the lumen.

12. The system of claim 7, wherein the cutting apparatus includes at least one cutting surface inclined relative to the axis in a first rotational direction.

13. The system of claim 11, further comprising an additional rotatable sampling element including:
an additional second cylindrical body concentrically disposed around the cylindrical body and supporting at least one additional cutting apparatus at a distal end, wherein the additional cutting apparatus includes at least one additional cutting surface inclined relative to the axis and facing an opposing rotational direction opposite to the first rotational direction, the cylindrical body being rotatable relative to the additional cylindrical body to scissor the tissue mass between the cutting surface and the additional cutting surface responsive to relative counter-rotation of the cylindrical body and the additional cylindrical body; and
an additional flexible drive shaft concentrically disposed around the flexible drive shaft and fixably engaged with a proximal end of the additional rotatable sampling element, wherein the additional flexible drive shaft is laterally movable with the flexible drive shaft and rotationally independent of the flexible drive shaft to enable counter-rotation of the cylindrical body relative to the additional cylindrical body.

14. The system of claim 13, wherein the actuator handle is configured to prevent the additional flexible drive shaft from rotating while the flexible drive shaft is rotated to enable the flexible drive shaft to rotate independently of the second flexible shaft.

15. The system of claim 13, wherein the actuator handle includes a counter-rotating mechanism mechanically couplable with the flexible shaft and the additional flexible shaft, wherein the counter-rotating mechanism is configured to cause the flexible shaft to rotate in a first direction while the additional flexible shaft is rotated in an opposite direction.

16. The system of claim 7, wherein the actuator handle includes an advancing mechanism configured to enable a portion of the handle to be moved to motivate the flexible shaft to linearly move the rotatable sampling element relative to the tissue mass.

17. The system of claim 7, further comprising a sheath configured to receive therein the rotatable sampling element and at least a portion of the flexible drive shaft and further configured to convey the rotatable sampling element adjacent the tissue mass.

18. The system of claim 17, wherein the actuator handle further includes a sheath actuator configured to linearly motivate the sheath relative to the tissue mass.

19. A method comprising:
positioning a rotatable sampling element adjacent a tissue mass;
advancing and rotating the rotatable sampling element such that a cutting apparatus cuts a tissue sample from the tissue mass; and
removably receiving the tissue sample into a receiving chamber.

20. The method of claim 19, further comprising:
positioning an additional rotatable sampling element circumferentially around the rotatable sampling element adjacent to a tissue mass; and
counter-rotating the rotatable sampling element and the additional rotatable sampling element to cut the tissue sample from the tissue mass.
